(19) United States
(12) Patent Application Publication
(10) Pub. No.: US 2010/0047293 A1
(43) Pub. Date: Feb. 25, 2010

(54) FRAGRANCE EMITTING PATCH

(76) Inventors: Elaine M. Gannon, Hoboken, NJ (US); David Lievin, Bois des Filion (CA); H. Michael Moscherosch, Doylestown, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/197,582

(22) Filed: Aug. 25, 2008

(51) Int. Cl. A61K 8/02; A61Q 13/00

(52) U.S. Cl. 424/401

(57) ABSTRACT

The present invention relates to a fragrance emitting patch that a user can attach to the body or an article of clothing, and more particularly to a fragrance emitting patch having a configuration that permits the easy application and removal of the fragrance emitting patch by a user.

### FIELD OF THE INVENTION

The present invention relates to a fragrance emitting patch that a user can attach to the body or an article of clothing, and more particularly to a fragrance emitting patch having a configuration that permits the easy application and removal of the fragrance emitting patch by the user.

### BACKGROUND OF THE INVENTION

Fragrance emitting devices are generally used to deliver a pleasant scent to the user. These devices have been used in the past to mask undesirable odors and can also be functionalized with an odor-controlling agent. The prior art discloses fragrance emitting patches that purport to deliver fragrance when a user attaches the patch onto their body or an article of clothing. These patches generally include one more or more layers of material, at least one of which is provided with a fragrance. Fragrance emitting patches generally include a positioning adhesive applied to an external surface of the patch for selectively adhering the patch onto the user's skin or article of clothing. Patches with multiple layers may also include a construction adhesive used to affix the layers of the patch to one another.

A problem with fragrance emitting patches of the type disclosed in the prior art is that such patches are often difficult to apply and/or difficult to remove. In particular, the user often finds it difficult to grasp the patch for attachment and/or removal and such prior art patches may tear upon removal of the patch from the body or garment. The inventors of the present invention have discovered a fragrance emitting patch construction that overcomes the shortcomings of the prior patches described above. In particular, fragrance emitting patches according to the present invention are easy to apply to a garment or the skin, remain securely attached to the garment or skin during use, and are easy to remove.

### SUMMARY OF THE INVENTION

In view of the foregoing, the present invention provides, a fragrance emitting patch including at least a first layer including a fragrance, a peripheral edge, a main body, a plurality of projections extending outwardly from the main body, wherein the portion of the peripheral edge located between adjacent projections of the plurality of projections defines an angle θ, each angle θ being between about 15° and about 130°.

### BRIEF DESCRIPTION OF THE DRAWINGS

FIG. 1 is a schematic view of a fragrance emitting patch in accordance with the invention adhered to an undergarment;

FIG. 2 is a bottom plan view of a fragrance emitting patch in accordance with the invention with the removable backing layer thereof partially torn away to reveal the positioning adhesive thereunder;

FIG. 3 is a cross sectional view of the patch shown in FIG. 2 taken along line 3-3 thereof;

FIG. 4 is a top plan view of a fragrance emitting patch in accordance with another embodiment of the invention;

FIG. 5 is a cross sectional view of the patch shown in FIG. 4 taken along line 5-5 thereof;

FIG. 6 is a top plan view of a fragrance emitting patch in accordance with yet another embodiment of the invention;

FIG. 7 is a cross sectional view of the patch shown in FIG. 6 taken along line 7-7 thereof;

FIGS. 8a-8e depict plan views of fragrance emitting patches having different shapes within the scope of the present invention;

FIG. 9 is a top plan view of a fragrance emitting patch according to the invention showing the inner radius R2 thereof;

FIG. 10 is a top plan view of the fragrance emitting patch depicted in FIG. 9, showing the outer radius R1 thereof; and

FIG. 11 is a top plan view of the fragrance emitting patch depicted in FIG. 9, showing the angle θ between adjacent projections of the patch.

### DETAILED DESCRIPTION OF THE INVENTION

As used herein, the term “construction adhesive” refers to any adhesive that is used to join two layers of material to one another.

As used herein, the term “positioning adhesive” refers to any adhesive that is used to removably attach a fragrance emitting patch to a user's skin or clothing.

In one embodiment of the invention, as shown in FIG. 1, the fragrance emitting patch according to the present invention is intended to be applied to a user's undergarment, such as a woman's panty, during use, to thereby provide a fresh scent to undergarment. The inventors have found that in order to achieve long lasting fragrance levels that can be detected from the perennial region of the user to the nose, a high amount of fragrance must be incorporated into the fragrance emitting patch. The inventors have found that users can detect a product including a fragrance applied to one of the layers of the patch in an add on amount of greater than 3 gsm (g/m²). In one embodiment of the invention, a fragrance is applied to at least one of the layers of the fragrance emitting patch in an amount of between about 3 gsm and about 15 gsm.

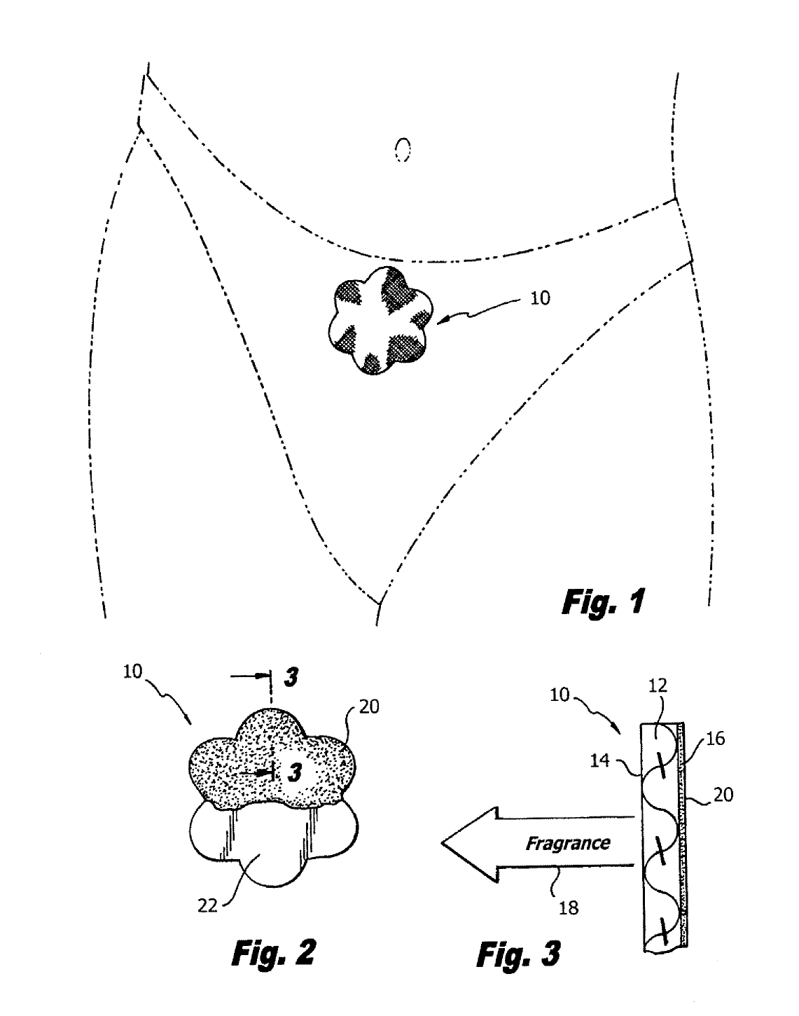

FIG. 1 illustrates an example of a fragrance emitting patch 10 according to the present invention, the patch 10 is adhered to the surface of a woman's undergarment to provide a fresh scent thereto. Referring to FIGS. 2 and 3, the patch 10 includes, according to a first embodiment of the invention, a porous primary layer 12 having a top surface 14 and a bottom surface 16. The primary layer 12 is provided with a fragrance represented by the numeral 18. The fragrance 18 may be applied to a top surface 14 or bottom 16 surface of the primary layer 12 or it may be infused into the primary layer 12 itself. The patch 10 is further provided with a positioning adhesive 20 applied to the bottom surface 16 of the primary layer 12. The positioning adhesive 20 allows a user to selectively apply the patch to a garment of clothing such as an undergarment, or directly to the skin.

The fragrance emitting patch 10 may be optionally provided with a removable backing layer 22, shown in FIG. 2, that is intended to protect the positioning adhesive 20 prior to use of the patch 10. The backing layer 22 may be constructed of a suitable paper and/or polymeric film material. The surface of the backing layer 22 in contact with the positioning adhesive 20 may be provided with a non-stick coating such as silicone to facilitate the removal of the backing layer 22 by the user prior to use.

According to one aspect of the invention, the primary layer 12 is constructed from a porous non-woven web material. The primary layer 12 may be composed of only one type of fiber, such as polyester or polypropylene or it may include a mixture of more than one fiber. The primary layer 12 may be composed of bi-component or conjugate fibers having a low melting point component and a high melting point component. The fibers may be selected from a variety of natural and synthetic materials such as nylon, polyester, rayon (in combination with other fibers), cotton, acrylic fiber and the like and combinations thereof. Preferably, the primary layer 12 has a basis weight in the range of about 10 gsm to about 75 gsm. Bi-component fibers may be made up of a polyester layer and a polyethylene sheath. Using a fusible fabric increases the ease with which the primary layer 12 may be mounted to any underlying layer should such an underlying layer be employed. According to another aspect of the invention the porous primary layer 12 is constructed from a microporous polymeric film material.

According to one aspect of the invention, the porous primary layer 12 is provided with the fragrance 18. The fragrance 18 may be selected from any common fragrances known to those of skill in the art. The fragrance 18 may also constitute a complex fragrance, i.e. a fragrance including a mixture of a number of different fragrance components. The fragrance 18 is preferably provided on or in the primary layer 12 in an amount greater than about 3 gsm (g/m²), preferably between about 3 gsm and about 15 gsm.

According to one aspect of the invention, the bottom surface 16 of the porous primary layer 12 is provided with a positioning adhesive 20. Preferably the positioning adhesive 20 is applied to the bottom surface 16 in an amount between about 8 gsm to about 25 gsm. Suitable positioning adhesive 20 compositions include hot melt adhesives based on block copolymers such as linear or radial co-polymer structures having the formula (A−B)ₓ wherein block A is a polyvinylarene block, block B is a poly(monoalkenyl) block, and x is an integer greater than or equal to one that denotes the number of polymeric arms. Suitable block A polyvinylarenes include, but are not limited to, polystyrene, polyalpha-methylstyrene, polyvinyltoluene, and combinations thereof. Likewise, suitable Block B poly(monoalkenyl) blocks include, but are not limited to, conjugated diene elastomers, such as polybutadiene, polyisoprene, and hydrogenated elastomers such as ethylene butylenes, ethylene propylene, polyisobutylene, or combinations thereof. Commercial examples of these types of block copolymers include Kraton™ elastomers from Kraton Polymers L.P, Vector™ elastomers from Dexco, SIBSTAR polymers from Kaneka USA, and Stereon™ from Firestone Tire & Rubber Co. Alternately, suitable acrylic hot melt adhesive polymers such as the ACResin hot melt adhesives from BASF Corp. may also be used. In addition to providing some level of insolubility to the fragrances, these systems can be rendered further insoluble via crosslinking using a UV radiation source.

Referring to FIGS. 6 and 7, a fragrance emitting patch 10a includes, according to another embodiment of the invention, a porous primary layer 12 having a top surface 14 and a bottom surface 16. The primary layer 12 is provided with a fragrance represented by the numeral 18. The fragrance 18 may be applied to a top surface 14 or bottom 16 surface of the primary layer 12 or it may be infused into the primary layer 12 itself. The patch 10 further includes a secondary layer 32 having a top surface 34 and bottom surface 36. The secondary layer 32 is adhered to a bottom surface 16 of the primary layer 12 by a construction adhesive 35 that is arranged between the layers 12 and 32. The bottom surface 36 of the secondary layer 32 is provided with the positioning adhesive 20 that permits a user to selectively apply the patch 10a to a garment of clothing such as an undergarment or directly to the skin.

The construction adhesive 35 is preferably selected from the same group of adhesives as the positioing adhesive 20. Thus suitable construction adhesive 35 compositions include hot melt adhesives based on block copolymers such as linear or radial co-polymer structures having the formula (A−B)ₓ wherein block A is a polyvinylarene block, block B is a poly(monoalkenyl) block, and x is an integer greater than or equal to one that denotes the number of polymeric arms. Suitable block A polyvinylarenes include, but are not limited to, polystyrene, polyalpha-methylstyrene, polyvinyltoluene, and combinations thereof. Likewise, suitable Block B poly(monoalkenyl) blocks include, but are not limited to, conjugated diene elastomers, such as polybutadiene, polyisoprene, and hydrogenated elastomers such as ethylene butylenes, ethylene propylene, polyisobutylene, or combinations thereof. Commercial examples of these types of block copolymers include Kraton™ elastomers from Kraton Polymers L.P, Vector™ elastomers from Dexco, SIBSTAR polymers from Kaneka USA, and Stereon™ from Firestone Tire & Rubber Co. Alternately, suitable acrylic hot melt adhesive polymers such as the ACResin hot melt adhesives from BASF Corp. may also be used. In addition to providing some level of insolubility to the fragrances, these systems can be rendered further insoluble via crosslinking using a UV radiation source. As shown in FIG. 7, the construction adhesive 35 is preferably applied to a bottom surface 16 of the primary layer 12 in an amount between 1 gsm and 25 gsm.

According to one aspect of the invention, the secondary layer 32 may be a non-porous layer. In one preferred embodiment of the invention, the secondary layer 32 is a non-porous polymeric film such as polyethylene or polypropylene film.

Alternatively, the secondary layer 32 may be a porous layer. A porous secondary layer 32 may be a nonwoven material composed of only one type of fiber, such as polyester or polypropylene or it may include a mixture of more than one fiber. The secondary layer 32 may be composed of bi-component or conjugate fibers having a low melting point component and a high melting point component. The fibers may be selected from a variety of natural and synthetic materials such as nylon, polyester, rayon (in combination with other fibers), cotton, acrylic fiber and the like and combinations thereof. Bi-component fibers may be made up of a polyester layer and a polyethylene sheath. Using a fusible fabric increases the ease with which the secondary layer 32 may be mounted to an adjacent layer, e.g. the primary layer 12. According to another aspect of the invention, the secondary layer 32 is constructed from a microporous polymeric film material.

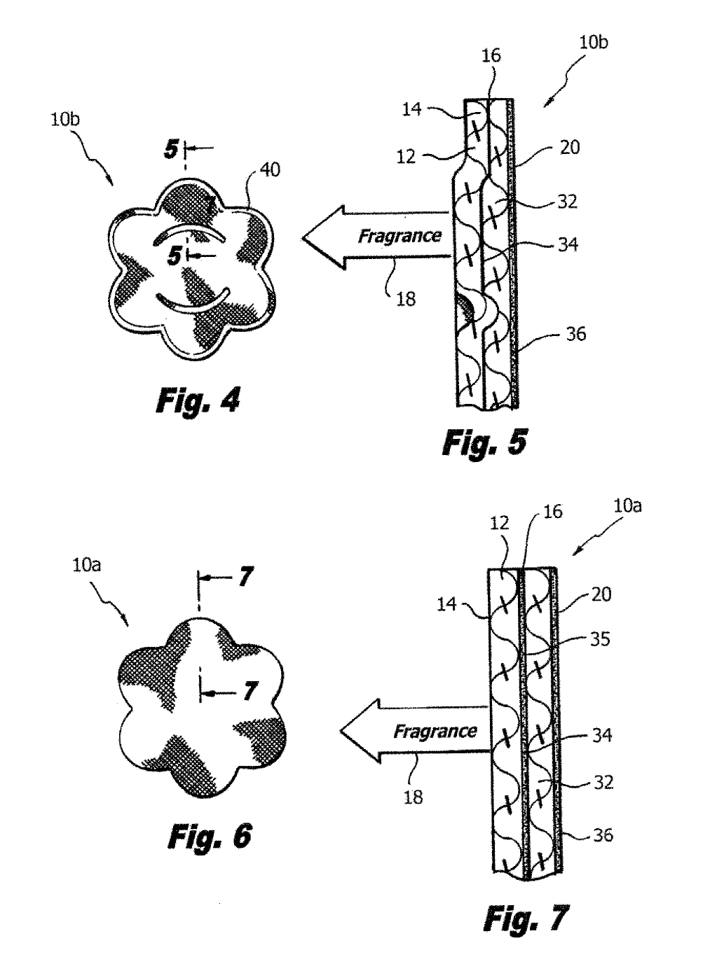

Referring to FIGS. 4 and 5, a patch 10b includes according to a yet another embodiment of the invention, a porous primary layer 12 having a top surface 14 and a bottom surface 16. The primary layer 12 is provided with a fragrance represented by the numeral 18. The fragrance 18 may be applied to a top 14 or bottom 16 surface of the primary layer 12 or it may be infused into the primary layer 12 itself. The patch 10b further includes a secondary layer 32 having a top 34 and bottom surface 36. The bottom surface 36 of the secondary layer 32 is provided with the positioning adhesive 20 that permits a user to selectively apply the patch to a garment of clothing such as an undergarment or directly to the skin.

In the embodiment of the invention shown in FIGS. 4 and 5 the primary layer 12 is secured to the secondary layer 32 in an adhesive free manner. For example, the primary layer 12 may be secured to the secondary layer by embossing using heat and pressure to fuse the primary layer 12 to the secondary layer 32. The primary layer 12 and secondary layer 32 may be constructed from nonwoven materials including heat fusible fibers to facilitate the bonding of the layers 12 and 32. In the embodiment shown in FIG. 4 and 5, the patch 10b includes a bonded area 40 around the periphery of the patch 10b. The patches 10, 10a and 10b described herein preferably have a thickness in the range of between about 0.25 mm and about 2.0 mm.

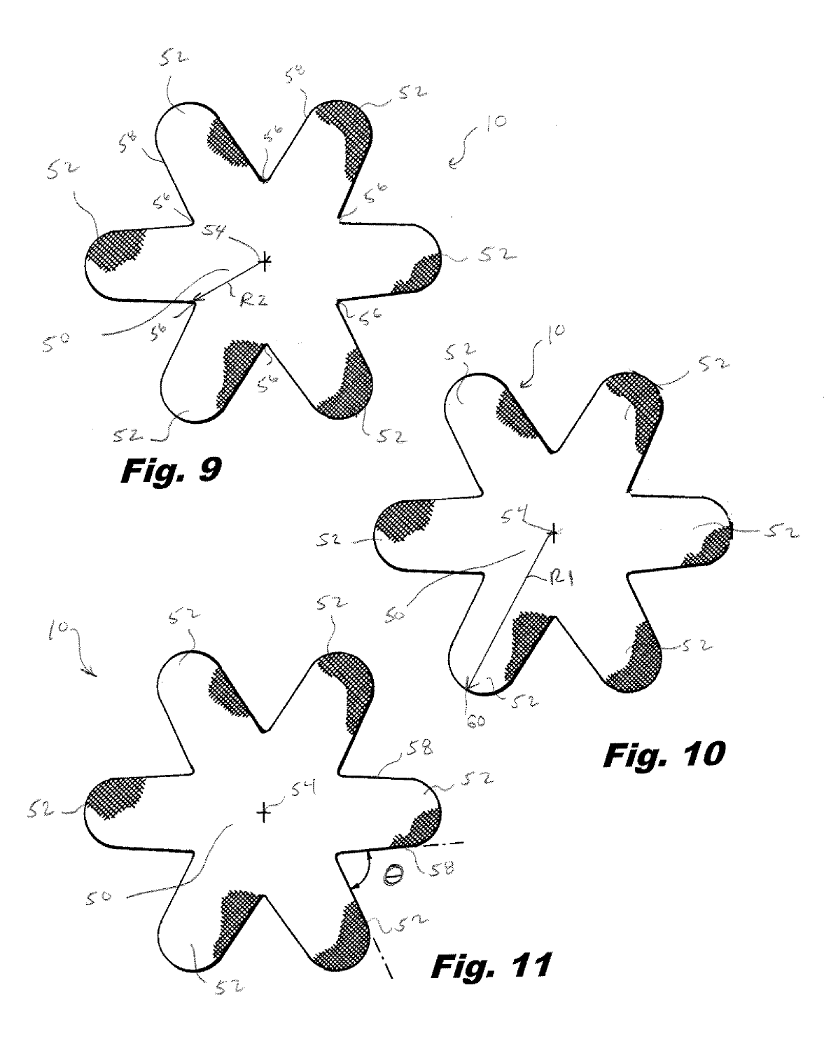

Referring to FIG. 9, fragrance-emitting patches 10 according to the present invention generally include a main body portion 50 and a plurality of projections 52 extending outwardly relative to the main body portion 50. Patches 10 according to the present invention preferably include between about 4 and about 8 projections 52.

In the particular embodiments of the invention shown in the Figures, the fragrance emitting patch is constructed in a flower or star-like shape having a plurality of petal portions extending outward from the main body portion, however other shapes are possible within the scope of the present invention provided that such shape includes a central body portion and a plurality projections extending outwardly therefrom in the manner described above.

As shown in FIGS. 9-11, fragrance emitting patches 10 according to the present invention have a center of gravity 54. The center of gravity 54 for a patch 10 having any given shape can be readily determined by any conventional method known to those of skill in the art. One simple method to determine the center of gravity of a flexible object, such as a fragrance emitting patch of the present invention, is to use the patch as a template and trace the shape of the patch onto cardboard or other light weight rigid material. The traced shape may then be cut from the rigid material. Once the rigid material is cut out, the location where the cut out shape can be balanced on top of a pointed object (such as a ball point pen) is the location of the center of gravity.

Referring to FIG. 9, fragrance emitting patches 10 according to the present invention have a plurality of juncture points 56, a juncture point 56 being defined herein as those locations on the peripheral edge 58 of the patch 10 where a projection 52 meets the main body portion 50 of the patch 10. As shown in FIGS. 9 and 10, fragrance emitting patches 10 according to the present invention preferably have an outer radius R1 and inner radius R2. As shown in FIG. 9, the inner radius (R2) for a patch 10 having a given shape is determined by measuring the distance from the center of gravity 54 of the patch 10 to the juncture point 56 located closest to the center of gravity 54. Patches 10 according to the present invention preferably have an R2 value in the range of about 7 mm to about 35 mm. As shown in FIG. 10, the outer radius (R1) for a patch 10 having a given shape is determined by measuring the distance from the center of gravity 54 to the location 60 on the peripheral edge 58 of the product located furthest away from the center of gravity 54. Patches according to the present invention preferably have an R1 value in the range of about 25 mm to about 35 mm. Patches 10 according to the present invention preferably have an R1/R2 ratio value in the range of about 1.0 to about 3.5. The inventors have discovered that patches 10 having the R1 and R2 values specified above, and the R1/R2 ratio specified above, are easy to handle and apply for the user and are also easy for the user to remove.

Although the patches 10 depicted in the Figures are symmetric, asymmetric shaped patches are considered within the scope of the invention provided that such shapes have the R1 and R2 values set forth above and the R1/R2 ratio set forth above. For asymmetric shapes the inner radius (R2) for a patch 10 having a given shape patch is determined by measuring the distance from the center of gravity 54 of the patch 10 to the juncture point 56 located closest to the center of gravity 54. The outer radius (R1) is determined by measuring the distance from the center of gravity 54 to the location 60 on the peripheral edge 58 of the product located furthest away from the center of gravity 54.

As shown in FIG. 11, the peripheral edge 58 of the patch 10 defines an angle θ between adjacent projections, preferably the angle θ is in the range of between about 15° and about 130°. The angle θ between projections can be measured with a simple protractor or with an imaging software like Image Pro Plus (Media Cybernetics Inc. Company, MD, USA). In this case, specimens have to be electronically scanned or a picture has to be taken with a digital camera and the angle measurement is made on the electronic picture.

The angle θ is formed by the two rays defined by the peripheral edge 58 of the patch sharing a common vertex located at one of the juncture points 56. The angle θ is measured along the two tangents defined by the peripheral edge portions that share the same vertex or intersection point.

It has been discovered by the inventors of the present application that a patch 10 having an angle θ between each projection 52 within the specified range helps prevent tearing of the patch 10 during application and removal of the patch by the user. It is noted that it is not required that every angle θ on a given patch be equal, i.e. the patch may be asymmetrical in shape, provided however that every angle θ falls somewhere within the range specified above.

### Method for Measuring Flexibility of a Fragrance Emitting Patch

The inventors of the present invention have discovered that fragrance emitting patches 10 according to the present invention are preferably highly flexible to thereby provide optimum comfort during use. In particular, patches 10 according to the present invention preferably have a modified circular bend stiffness (MCB) less than 1200 g according to the test method set forth below. Preferably fragrance meeting patches according to the present invention have an MCB stiffness in the range of about 250 g to about 650 g.

Prior to testing, all patches should be conditioned for 2 hours at (21±1)° C. and (50±2) % R.H. (relative humidity). Flexibility is determined by a test that is modeled after the ASTM D 4032-82 CIRCULAR BEND PROCEDURE, the procedure being considerably modified and performed as follows. The CIRCULAR BEND PROCEDURE is a simultaneous multi-directional deformation of a material in which one face of a specimen becomes concave and the other face becomes convex. The CIRCULAR BEND PROCEDURE gives a force value related to flexural resistance, simultaneously averaging stiffness in all directions.

The apparatus necessary for the CIRCULAR BEND PROCEDURE is a modified Circular Bend Stiffness Tester, having the following parts:
A smooth-polished steel plate platform, which is 84.33 mm in diameter and has an 18.75 mm diameter orifice. The lap edge of the orifice should be at a 45 degree angle to a depth of 4.75 mm;
A plunger having an overall length of 70.7 mm. A diameter of 17.0 mm, a ball nose having a radius of 8.51 mm and a needle point extending 0.88 mm there from having a base of 0.25 mm base, the plunger being mounted concentric with the orifice and having equal clearance on all sides. Note that the needlepoint is merely to prevent lateral movement of the test specimen during testing. The bottom of the plunger should be set well above the top of the orifice plate. From this position, the downward stroke of the ball nose is to the exact bottom of the plate orifice;
A force-measurement gauge and more specifically a MTS load cell. The load cell has a load range of from about 0.0 to about 2500.0 g;
An actuator and more specifically the MTS model RT/1 having a load cell. The MTS model RT/1 is made by MTS Systems Corporation, Eden Prairie, Minn.

In order to perform the procedure for this test, as explained below, five representative product samples for each patch to be tested are necessary. The location of the patch to be tested is the center of gravity of the patch. Any release paper or packaging material is removed from the product sample and any exposed adhesive, such as garment positioning adhesive, is covered with a non-tacky powder such as talc or the like. The talc should not affect the MCB measurement.

The test specimens should not be folded or bent by the test person, and the handling of specimens must be kept to a minimum and to the edges to avoid affecting flexural-resistance properties.

A test specimen is centered on the orifice platform below the plunger such that the non-woven or scented facing layer of the test specimen is facing the plunger and the barrier layer of the specimen is facing the platform. The plunger speed is set at 50.0 cm per minute per full stroke length. The indicator zero is checked and adjusted, if necessary. The plunger is actuated. Touching the test specimen during the testing should be avoided. The maximum force reading to the nearest gram required to force the whole specimen to go in the orifice is recorded. The above steps are repeated until all of five test specimens have been tested. An average is then taken from the five test values recorded to provide the modified circular bend stiffness (MCB).

### Method for Measuring the Peel Strength of a Fragrance Emitting Patch

Inventors of the present invention have discovered that to ensure the secure attachment of a patch 10 during use and also permit the easy removal of the same by the user, the patch 10 preferably has a peel strength in the range of between about 100 g to about 1000 g. A method for measuring the peel strength of a fragrance emitting patch is set forth below.

The peel strength test method measures the force needed to separate the patch from a standard cotton fabric. The set-up required for this measurement consists of 2 adjustable pneumatic jaws of 7.62 cm (3 inches) wide or more mounted on an Instron Tensiometer Tester model 1123 or a similar instrument. The Tensiometer is equipped with a load cell of 2000 g or more. A pneumatic air bag apparatus capable of applying 2.5 psi (70 inches of H₂O) of gauge pressure is needed to prepare specimens.

In order to perform the procedure for this test, five test specimens for a patch having a given shape are required. A standard cotton fabric is cut slightly wider and 1 inch longer than the patch and laid flat on a table. The release papers are removed from the patch and the patch is applied on the cotton fabric in a way such that the patch is flat on the cotton fabric and one projection is oriented in the vertical direction away from the fabric. The patch is arranged such that the positioning adhesive is arranged in surface to surface contact with the cotton fabric. Light pressure is manually applied to secure the patch to the cotton fabric. Two pieces of adhesive tape, such as a common masking tape, are secured to the vertically arranged projection on opposed surfaces thereof and the adhesive tape pieces are secured between the upper jaws of the apparatus. Then, the specimen is placed in the air bag apparatus in a horizontal position with the standard cotton strip facing upwards and a pressure of 70 inches of H₂O is applied for 2 minutes. The distance between the movable jaws of the Tensiometer is set to 1.0 inch (indicator zero) and the specimen is secured within the jaws of the Tensiometer. Specifically, the cotton fabric is secured within the lower jaws of the apparatus and the adhesive tape pieces are secured within in the upper jaws. The crosshead speed of the Tensiometer is set at 100.0 cm per minute. Touching the test specimen during the testing should be avoided. The patch is completely removed form the cotton fabric and the maximum force or the peak load reading to the nearest gram is recorded. The above steps are repeated until all of five test specimens have been tested. An average is then taken from the five test values recorded to provide average peel strength.

### Method for Measuring Tear Strength of a Fragrance Emitting Patch

Inventors of the present invention have discovered that to ensure that a fragrance emitting patch 10 does not tear upon removal by the user the fragrance emitting patch must have a tear strength greater than the peel strength of the patch according to the test method set forth above. Fragrance emitting patches according to the present invention preferably have a tear strength in the range from about 650 g to about 2000 g. A method for measuring the tear strength of a fragrance emitting patch is set forth below.

The tear strength test method gives the force needed to tear or break a projection from the main body of the patch. If the tear strength is lower than the peel strength, it is not possible to remove the patch from a garment or the body without tearing the patch.

The set-up required for this measurement consist of 2 pneumatic jaws of 7.62 cm (3 inches) wide or more mounted on an Instron Tensiometer Tester model 1123 or a similar instrument. The Tensiometer is equipped with a load cell of 2000 g or more.

In order to perform the procedure for this test, five test specimens for a patch having a given shape are required. Any release paper or packaging material is removed from the product sample and any exposed adhesive, such as garment positioning adhesive, is covered with a non-tacky powder such as talc or the like. The talc should not affect the tear strength measurement.

Two pieces of adhesive tape, such as common masking tape, are secured to one of the projections of the patch on opposed surfaces thereof. The distance between the movable jaws of the Tensiometer is set to 1.0 inch or more, depending on the patch size (indicator zero) and the specimen is placed between the jaws of the Tensiometer. The main body of the patch is secured within the lower jaws and the adhesive tape pieces are secured within the upper jaws of the apparatus. The crosshead speed of the Tensiometer is set at 100.0 cm per minute. Touching the test specimen during the testing should be avoided. The projection is completely removed form the main body of the specimen, and the maximum force or the peak load reading to the nearest gram is recorded. The above steps are repeated until all of five test specimens have been tested. An average is then taken from the five test values recorded to provide average tear strength.

### INVENTIVE EXAMPLES 1-5

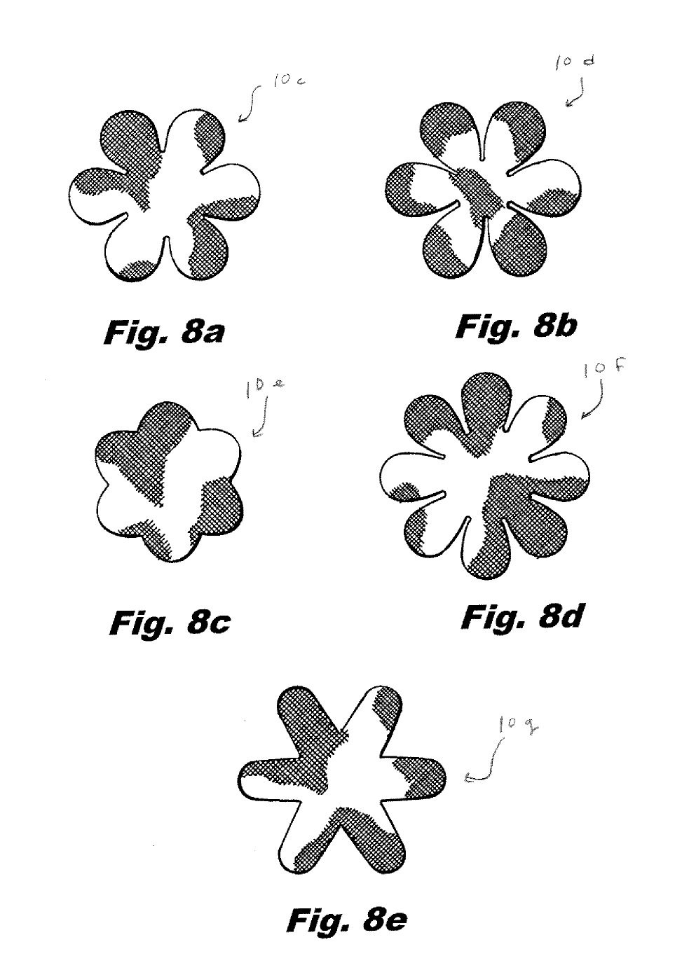

Fragrance emitting patches according to the present invention were constructed in each of the shapes 10c, 10d, 10e, 10f and 10g shown in FIGS. 8a-8e respectively. Each of the patches were constructed from a 40 gsm nonwoven primary layer formed from 50% by weight polypropylene fibers and 50% by weight polyester fibers, and a 30 gsm polyethylene film secondary layer. The primary layer was adhered to the secondary layer using a conventional construction adhesive. A layer of positioning adhesive (H.B. Fuller Company, Saint Paul, Minn., product code NW1042) was applied to the bottom surface of the secondary layer and a conventional fragrance was applied to the top surface of the primary layer. The inventive samples were tested according to the test methods set forth above, the results of which are set forth in the table provided below.

TABLE

| | | | | MCB | Peel | Tear | | |
|---|---|---|---|---|---|---|---|---|
| | R1 | R2 | | Stiffness | Strength | Strength | # of | Angle |
| | (mm) | (mm) | R1/R2 | (g) | (g) | (g) | Projections | θ (°) |
| Inventive | 34.54 | 15.27 | 2.26 | 604 | 633 | 1098 | 6 | 20 |
| Example | | | | | | | | |
| #1 | | | | | | | | |
| Inventive | 34.54 | 10.16 | 3.4 | 303 | 662 | 673 | 6 | 20 |
| Example | | | | | | | | |
| #2 | | | | | | | | |
| Inventive | 25 | 20 | 1.25 | 1141 | 239 | 1857 | 6 | 110 |
| Example | | | | | | | | |
| #3 | | | | | | | | |
| Inventive | 34.54 | 15.24 | 2.27 | 464 | 508 | 1059 | 6 | 70 |
| Example | | | | | | | | |
| #4 | | | | | | | | |
| Inventive | 34.54 | 15.24 | 2.27 | 457 | 884 | 1164 | 8 | 20 |
| Example | | | | | | | | |
| #5 | | | | | | | | |

In view of the above fragrance emitting patches according to the present invention provide a unique combination of properties that permits the easy application and removal of the fragrance emitting patch by the user, while at the same time insuring that the patch remains securely in place during use.

We claim:

1. A fragrance emitting patch comprising:
at least a first layer including a fragrance;
a peripheral edge;
a main body;
a plurality of projections extending outwardly from the main body;
wherein the portion of the peripheral edge located between adjacent projections of the plurality of projections each an angle θ, each angle θ being between about 15° and about 130°.

2. The fragrance emitting patch according to claim 1, wherein the patch has an R1/R2 ratio in the range from about 1.0 to about 3.5.

3. The fragrance emitting patch according to claim 1, wherein R1 is in the range of about 25 mm to about 35 mm and R2 is in the range from about 7 mm to about 25 mm.

4. The fragrance emitting patch according to claim 3, wherein the patch has an MCB stiffness less than 1200 g.

5. The fragrance emitting patch according to claim 4, wherein the patch has a peel strength in the range of about 100 g to about 1000 g.

6. The fragrance emitting patch according to claim 5, wherein the patch has a tear strength in the range of about 650 g to about 2000 g.

7. The fragrance emitting patch according to claim 6, wherein the patch has between about 4 and about 8 projections.

8. The fragrance emitting patch according to claim 7, wherein the tear strength of the patch is greater than the peel strength of the patch.
